(19) United States
(12) Patent Application Publication
(10) Pub. No.: US 2016/0120204 A1
(43) Pub. Date: May 5, 2016

(54) METHOD FOR PACKAGING AND DELIVERING SHEA BUTTER AND COCOA BUTTER

(71) Applicant: Anna Premazon, Seal Beach, CA (US)

(72) Inventor: Anna Premazon, Seal Beach, CA (US)

(21) Appl. No.: 14/926,337

(22) Filed: Oct. 29, 2015

Related U.S. Application Data
(60) Provisional application No. 62/072,674, filed on Oct. 30, 2014.

(51) Int. Cl. A23G 1/00; A61K 36/185; A61K 8/92; B65B 63/08

(52) U.S. Cl. CPC A23G 1/0066; B65B 63/08; A61K 36/185; A61K 8/922; A61K 2800/87

(57) ABSTRACT

The invention is a method for processing bulk form shea butter and cocoa butter for individual home use, by preparing molds of recyclable or non-recyclable, grease proof and leak proof paper, where the paper can be used in a microwave or conventional oven, or directly without modification, and when filled the mold will have a weight of between two ounces and five pounds, that can then be packaged.

### CROSS-REFERENCES

Provisional patent application filed Oct. 30, 2014, No. 62/072,674.

I have invented a method for packaging, delivering and manufacturing shea and cocoa butter into individual, reusable and recyclable paper molds that can be heated in the microwave or conventional oven.

### STATEMENT REGARDING FEDERALLY-SPONSORED RESEARCH OR DEVELOPMENT

None.

### NAMES OF PARTIES TO JOINT RESEARCH AGREEMENT

None.

### REFERENCE TO “SEQUENCE LISTING”

None.

### SUMMARY OF INVENTION

Shea butter has many applications, for instance, it is a significant ingredient in cosmetics, shampoos, in topical skin and lip care, as a moisturizer, and for healing of cuts and burns. Shea butter is the fatty substance derived by crushing nuts from the shea tree also known as the karite tree. It contains important vitamins that nourish the skin. Vitamins A and E help maintain the skin and keep it clear and healthy. They are particularly helpful for sun damaged skin. They help prevent premature wrinkles and facial lines. Vitamin F acts as a skin protector and rejuvenator.

Cocoa butter is used in making chocolate and as an ingredient in other food preparations. It is widely used in ointments, toiletries, and pharmaceuticals. It is a vegetable fat derived from the cocoa bean. Cocoa butter is a natural moisturizer that melts at room temperature which makes it ideal for your skin. Its health benefits come from its cocoa mass polyphenol. Cocoa butter, also called theobroma oil or theobroma cacao, is a pale-yellow, pure, edible vegetable fat extracted from the cocoa bean.

Although, shea butter and cocoa butter are different compounds, they can be packaged for delivery to individual consumers for a variety of uses.

Both, raw shea butter and raw cocoa butter are processed and then imported to be sold by manufacturers in bulk. In a raw format, shea butter and cocoa butter are typically sold in bulk for wholesale uses by distributors. The raw format involves delivery in large quantities, which is not compatible with home or individual consumer use. There is a long felt need to acquire shea butter and cocoa butter in smaller and individualized sticks or bars, which can then be heated, melted and mixed with other components for home use as contrasted with customary, single bin or brick packaging that requires the butters to be removed in order to work with the compound.

### BRIEF DESCRIPTION OF DRAWINGS

FIG. 1 is an oblique view looking downward on the top of the paper mold as the liquid shea butter or alternatively, liquid cocoa butter, is being poured into the mold.

FIG. 2 is an oblique side view showing the liquid hardening within the paper mold.

FIG. 3 shows the paper mold being removed from the hardened stick.

### DESCRIPTION OF THE INVENTION

Shea butter can be readily acquired by an importer or in solid bulk from a manufacturer or processor. The description on manufacturing shea butter that is acquired in solid bulk for individual use is the same as for manufacturing cocoa butter, except as noted below. For convenience, the references below are to shea butter only, but apply equally to cocoa butter.

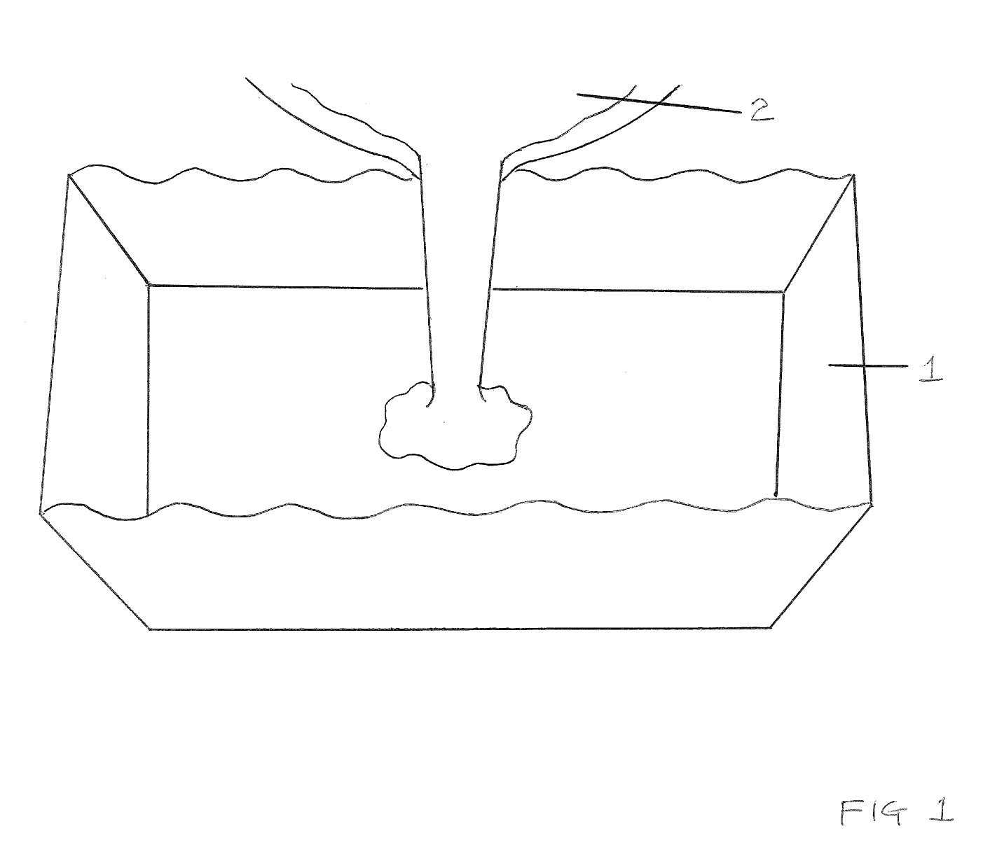

The steps to packaging, delivering and manufacturing shea butter for household use comprise, first molds 1, FIG. 1 are prepared. The molds have individual compartments shaped in the form of a rectangular solid or cuboid or ball or star or other configuration, at varying weights. In FIG. 1 a rectangular solid mold is shown, however, other mold forms are also considered to be part of this invention. The volume of the individual compartments is sufficient to accommodate a two, four, six, eight, or sixteen ounces - up to five pounds, stick, bar or ball (for convenience, collectively referred to as “stick”).

Second, the molds 1 are made from paper. This paper can be recyclable or non-recyclable and is grease proof and leak proof. The paper molds can go into a microwave or conventional oven to heat the shea butter. The paper mold isolates the shea butter sticks, permitting easy removal after cooling. The paper mold also serves as the holder for the shea butter determining its shape and size. In a preferred embodiment, the molds are made 100% from paper, that is fully recyclable. Colored paper can also be used and individually branded.

Third, the solid bulk raw shea butter is melted and carefully poured as a liquid 2, into the molds. The melting temperature for shea butter is between 89 to 113 degrees Fahrenheit. The melting temperature for cocoa butter is between 93 to 101 degrees Fahrenheit. There is a wax paper or plastic wrap top (not shown in the drawings) that keeps contaminants from falling into the shea butter and keeps it off hands or tools while handling the sticks—it is then wrapped with cellophane to keep the shea butter sealed.

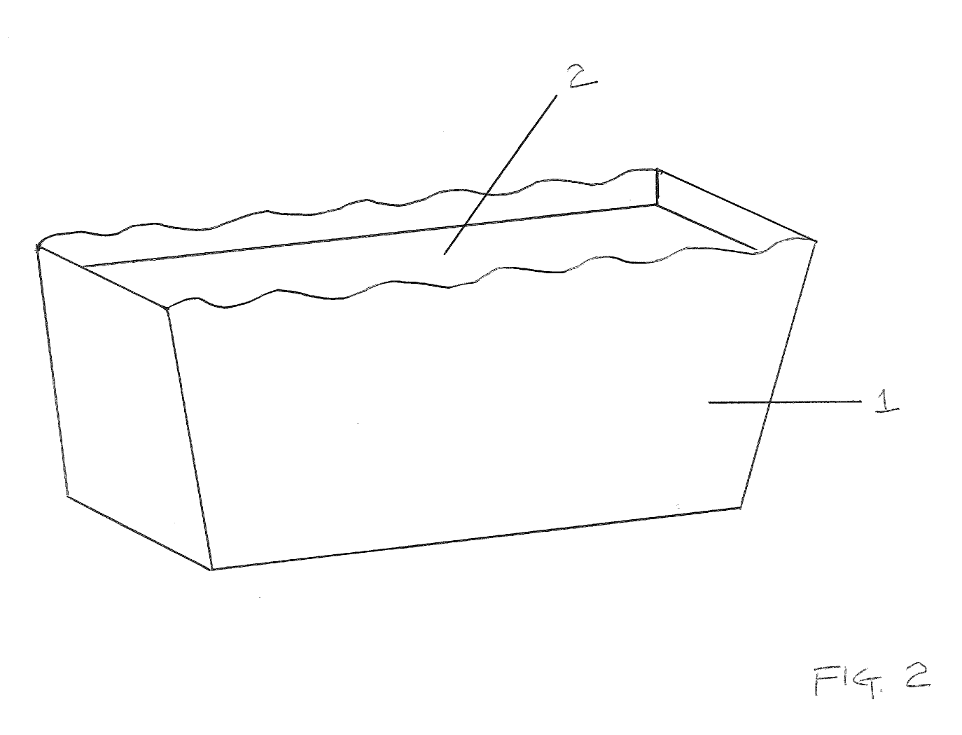

Fourth, the shea butter is permitted to cool in the mold as shown in FIG. 2. Cooling time may vary depending on the intended use for a product in which the shea butter is an ingredient. After cooling and then hardening as a solid, one or more of the shea butter sticks within the paper molds can be packaged for use.

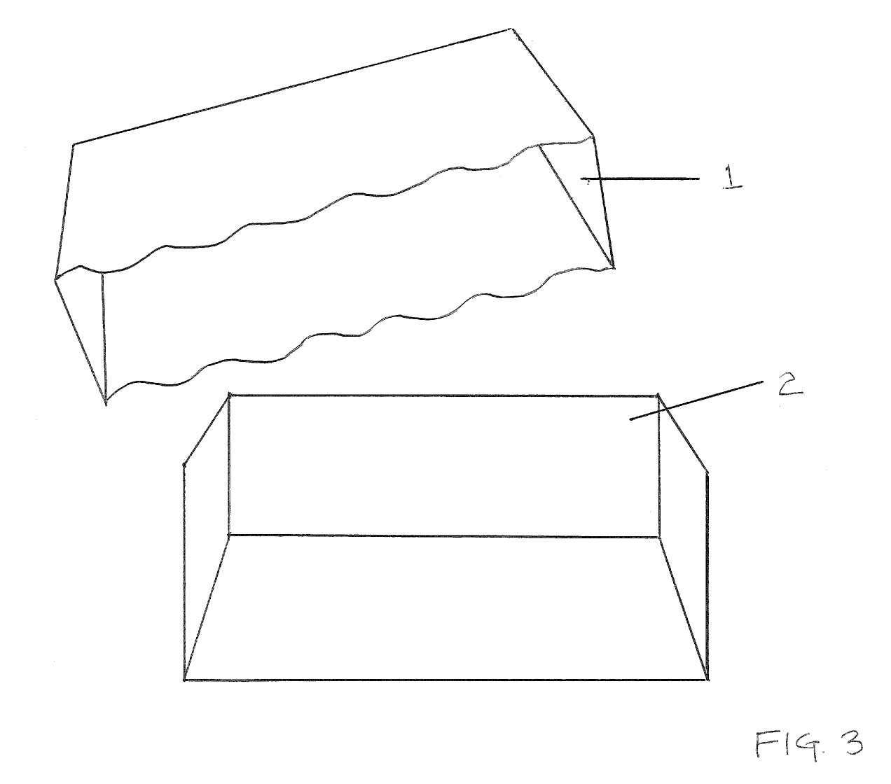

Fifth, the shea butter in each mold can be removed or left inside the mold to then be heated in the oven or microwave to be used as shown in FIG. 3. The shea butter in the mold is now ready for home use in products such as homemade soaps, lotions, or lip balms, as well as for household homeopathic recipes. In addition, this form of shea butter can be used directly as a product for skin condition.

The above description of the preferred embodiment of the present invention has been presented for the purposes of illustration and description. It is not intended to be exhaustive or to limit the invention to the precise form disclosed. Many modifications and variations are possible in light of the above teachings. It is intended that the scope of the present invention not be limited by this detailed description, but by the claims and the equivalents to the claims.

I claim:

1. A method for packaging and delivering shea butter for individual use comprising:
acquiring bulk shea butter,
preparing molds of recyclable grease proof and leak proof paper, where the paper can be heated by microwave and conventional oven, and when filled with shea butter the mold will have a weight of between two ounces and five pound,
melting the shea butter,
pouring the melted shea butter into the molds,
allowing the melted butter to cool, and
then packaging the molded shea butter for household use.

2. The method of claim One, where non-recyclable grease proof and leak proof paper is used.

3. A method for packaging and delivering cocoa butter for individual use comprising:
acquiring solid bulk cocoa butter,
preparing molds of recyclable grease proof and leak proof paper, where the paper can be heated by a microwave and conventional oven, and when filled with cocoa butter the mold will have a weight of between two ounces and five pounds,
melting the cocoa butter,
pouring the melted cocoa butter into the molds,
allowing the melted butter to cool, and
then packaging the molded cocoa butter for household use.

4. The method of claim Three, where non-recyclable grease proof and leak proof paper is used.
